(19) United States
(12) Patent Application Publication
(10) Pub. No.: US 2020/0181565 A1
(43) Pub. Date: Jun. 11, 2020

(54) Bacterial Growth on Non-Animal Derived Media

(71) Applicant: Inventprise, LLC, Redmond, WA (US)

(72) Inventors: Subhash V. Kapre, Redmond, WA (US); Seshu K. Gudlavalleti, Atlanta, GA (US)

(73) Assignee: Inventprise, LLC, Redmond, WA (US)

(21) Appl. No.: 16/703,511

(22) Filed: Dec. 4, 2019

Related U.S. Application Data
(60) Provisional application No. 62/775,987, filed on Dec. 6, 2018.

(51) Int. Cl. C12N 5/00; C12N 1/20

(52) U.S. Cl. CPC C12N 5/0043; C12N 5/0031; C12N 1/20; C12N 2500/14; C12N 2500/16; C12R 1/46; C12N 2500/34; C12N 2500/24; C12N 2500/74; C12N 2500/76; C12N 2500/32

(57) ABSTRACT

The invention is directed to tools, compositions, and methods for the cultivation of microorganisms in culture media that is devoid of animal-derived materials such as blood, and, in particular, to compositions of meat-free media.

### REFERENCE TO RELATED APPLICATIONS

This application claims priority to U.S. Provisional Application No. 62/775,987 filed Dec. 6, 2018, the entirety of which is hereby incorporated by reference.

### BACKGROUND

### 1. Field of the Invention

The invention is directed to tools, compositions, and methods for the cultivation of microorganisms in culture media that is devoid of animal-derived materials such as blood, and, in particular, to compositions of meat-free media.

### 2. Description of the Background

Microorganisms from the environment, infected tissues, biological specimens, derived from tissues, or genetically engineered are cultured and maintained in laboratory settings for a variety of reasons including but not limited to for diagnostic or identification purposes, for growth and propagation (e.g., of the cells or infections within cells such as virus or bacteria that may be present), and for cloning. Cell culture comprising a collection of techniques to maintain or grow cells, tissues or organs under sterile conditions on a nutrient culture medium of known composition. Tissue culture is widely used to produce clones in a method known as micropropagation. Microorganism such as bacterial and genetically modified microorganisms are often cultured for identification of a particular strain or serotype, or for testing sensitivity or resistance to various chemical compounds such as antimicrobials (e.g., antibiotics).

In all instances, growth of microorganisms is carefully controlled and monitored to ensure replicability and to obtain meaningful results. Growth media may be liquid or solid with solid media in the form or a semisolid such as agar. Each medium, in whatever form, must contain the essential nutrients required by the particular microorganism. The essential nutrients, as distinguished from non-essential, are those chemical compounds that the cell is unable to make for itself, and therefore must obtain directly from its environment. For liquid media, various types of animal serum (e.g., fetal bovine serum, horse serum, goat serum) are included to provide those essentials and for solid media, the essential components are provided by animal extracts (e.g., beef extract). Animal serums and extracts are expensive and never precisely uniform in their constituents. However, propagation of many different types of cells requires animal products, such as blood, for growth. Having a non-animal derived growth media would lower costs and help to standardize testing and experimentation.

### SUMMARY OF THE INVENTION

The present invention overcomes the problems and disadvantages associated with current strategies and designs and provides new devices and methods of media for microorganism maintenance and propagation that requires no animal products.

One embodiment of the invention is directed to compositions comprising one or more salts; a magnesium salt; a calcium salt; soy meal; a polysaccharide; at least two amino acids; yeast extract; a ferrous or ferric salt; and pyruvate. Preferably the one or more salts comprise sodium chloride. Preferably the magnesium salt comprises magnesium chloride or magnesium sulfate. Preferably the calcium salt comprises calcium chloride or calcium sulfate. Preferably the soy meal comprises an enzymatic hydrolysate of soy meal. Preferably at least two amino acids comprise cysteine and thiamine. Preferably the saccharide comprise glucose. Preferably the ferrous or ferric salt comprises ferrous sulfate, ferric citrate, or both. Preferably the composition comprises an aqueous solution or a dry powder. Preferably the aqueous solution comprises from about 1-5 g/L of the one or more salts; from about 0.1 to 2.0 g/L of the magnesium salt; from about 0.001 to 0.1 g/L of the calcium salt; from about 2-10 g/L of the soy meal; from about 5-20 g/L of the polysaccharide; from about 0.001 to 0.1 g/L of the at least two amino acids; from about 1-10 g/L of the yeast extract; from about 0.0001-0.001% of the ferrous or ferric salt; and from about 0.01-1.0% of the pyruvate.

Another composition of the invention comprises one or more salts; soy meal; a saccharide; yeast extract; a plant protein hydrolysate, a ferrous or ferric salt; and pyruvate. Preferably the one or more salts comprises sodium chloride. Preferably the soy meal comprises an enzymatic hydrolysate of soy meal. Preferably the saccharide comprise glucose. Preferably the yeast extract comprises a vegetable yeast extract. Preferably the plant protein hydrolysate comprises atholate. Preferably the ferrous or ferric salt comprise ferrous sulfate or ferric citrate. Preferably the enzymatic hydrolysate of soy meal is at a concentration of from about 0.5-10%, the polysaccharide is at a concentration of from about 0.5-5%, the vegetable yeast extract is at a concentration of from about 0.1-10%, the plant protein hydrolysate is at a concentration of from about 1-10%, the ferrous or ferric salt is at a concentration of from about 0.001-0.01%, and the pyruvate is at a concentration of from about 0.01-1.0%. Preferably a composition of the disclosure is an aqueous solution, a dry powder, or a semi-solid such as agar.

Another embodiment of the invention is directed to methods for culturing a microorganism comprising: obtaining a sample of the microorganism; and contacting the microorganism to a medium comprising a composition of the invention. Preferably the microorganism comprises Streptococcus pneumoniae.

Other embodiments and advantages of the invention are set forth in part in the description, which follows, and in part, may be obvious from this description, or may be learned from the practice of the invention.

### DESCRIPTION OF THE INVENTION

Microorganisms such as Streptococcus pneumoniae are conventionally cultivated on blood agar plates. These types of bacteria do not grow on a media without animal blood. The requirement for animal blood in the growth medium does not allow for exact standardization, increase costs, and supply is often limited. Moreover, although blood and blood products may be certified, certification does not guarantee that agents such TSE (Transmissible Spongiform Encephalopathies (e.g., Bovine Spongiform Encephalopathy (BSE)) will not be present.

Media has been surprisingly discovered that will maintain the growth and propagation of microorganisms in culture media that is completely free of animal products. The absence of animal products in culture media substantially reduces costs and provides for increased standardization and a substantially increased safety. In addition, animal-free media as disclosed herein provided up to equivalent growth as compared to growth of the same microorganisms on blood agar plates.

One embodiment of the invention is directed to animal-free media for the growth and propagation of various species of Streptococcus including Streptococcus pneumoniae. As the media contains no animal products, it contains no blood, blood products or serum obtained or derived from animals including humans. Preferably the animal products excluded are products of mammals and include fetal animals, or young or older animals. Typical animal serums include, for example, bovine serum (e.g., fetal bovine serum), caprine serum, equine serum and products obtained from such animals.

The composition comprises one or more salts; a magnesium salt; a calcium salt; soy meal; a saccharide; at least two amino acids; yeast extract; a ferrous or ferric salt; and pyruvate. Preferably the one or more salts comprise sodium chloride, sodium sulfate, potassium chloride, or potassium sulfate, at a working concentration of from about 1-5 g/L. Preferably the magnesium salt comprises magnesium chloride or magnesium sulfate, at a working concentration of from about 0.1 to 2.0 g/L. Preferably the calcium salt comprises calcium chloride or calcium sulfate at a working concentration of from about 0.001 to 0.1 g/L. Preferably the soy meal comprises an enzymatic hydrolysate of soy meal, such as for example atholate, at a working concentration of from about 2-10 g/L. Preferably the saccharide comprises glucose, dextrose, sucrose, fructose, or a modified or substituted polysaccharide, at a working concentration of from about 5-20 g/L. Preferably the at least two amino acids comprise cysteine and thiamine, at a combined working concentration of from about 0.001 to 0.1 g/L. Preferably the yeast extract is at a working concentration of from about 1-10 g/L. Preferably the ferrous or ferric salt comprises ferrous sulfate or ferric citrate, at a working concentration of from about 0.0001-0.001%. Preferably the pyruvate comprises sodium pyruvate at a working concentration of from about 0.01-1.0%.

Another preferred composition comprises one or more salts; soy meal; a saccharide; yeast extract; a plant protein hydrolysate, a ferrous or ferric salt; and pyruvate. Preferably the one or more salts comprise sodium chloride, sodium sulfate, potassium chloride, or potassium sulfate, at a working concentration of from about 0.5-4%. Preferably the soy meal comprises an enzymatic hydrolysate of soy meal, such as for example SoyTone, at a working concentration of from about 0.5-10%. Preferably the saccharide comprises glucose, dextrose, sucrose, fructose, or a modified or substituted saccharide, at a working concentration of from about 0.5-5%. Preferably the vegetable yeast extract comprises, such as for example a vegetable yeast extract, at a working concentration of from about 0.1-10%. Preferably the plant protein hydrolysate comprises atholate at a working concentration of from about 1-10%. Preferably the ferrous or ferric salt comprises ferrous sulfate or ferric citrate, at a working concentration of from about 0.001-0.01%. Preferably the pyruvate comprises sodium pyruvate at a working concentration of from about 0.01-1.0%.

The compositions as disclosed herein may be maintained at ambient temperatures for extended periods of time as a dry powder (e.g., lyophilized), a liquid composition, or as a semi-solid (e.g., agar). The period of time may be, for example, weeks, months or years. Preferably the composition is prepared sterilely by sterile filtration, heat sterilization, sterile irradiation, or a combination thereof. Dry power is preferably mixed with an agar or other stable support for preparation of agar plates or maintained as a liquid medium. The percent agar of the composition is determined by one of ordinary skill in the art from the specific characteristics of the microorganism.

Another embodiment of the invention is directed to method for culturing and propagating microorganisms by contacting an organism with a composition disclosed herein. A preferred microorganism comprises a Streptococcus sp. (e.g., Streptococcus pneumoniae), Staphylococcus sp. (e.g., Staphylococcus aureus), Pseudomonas sp. (e.g., Pseudomonas aeruginosa), Escherichia sp. (e.g., Escherichia coli), Shigella sp., Salmonella sp., Neisseria sp., and combinations thereof. The specific growth conditions for each are well known to those skilled in the art and, accordingly, compositions of the invention may include various additional non-animal derived ingredients for maximal grown of the desired microorganisms.

The following examples illustrate embodiments of the invention, but should not be viewed as limiting the scope of the invention.

### EXAMPLES

### Example 1 Growth of Streptococcus pneumoniae

Twenty strains of Streptococcus pneumoniae in glycerol stocks (PNU) were obtained, namely serotypes 1, 2, 3, 4, 5, 6A, 6B, 7F, 8, 9V, 12F, 14, 15B, 18C, 19A, 19F, 22F, 23F, and 33F, and tested for their ability to grow on selected defined iron supplemented Pneumococcal meat-free (MF) media on both agar plates and in liquid broth. Media was identified that would facilitate seed bank preparation, thus avoiding blood agar passages. Two meat-free (MF) media are referred to as PNU-Fe and SoyTone-Fe. SoyTone is an enzymatic digestion of soy meal (commercially available from VWR corporation, USA). Polysaccharide yields of MF media cultivated strains were compared by partial down-stream purification of supernatants generated by mini-bioreactor fermentation batches.

Working from the glycerol stocks, the twenty strains were used to culture Trypticase Soy Agar (TSA) with 5% sheep blood agar plates. From blood agar plates each strain was passaged for three consecutive days on both PNU-Fe and SoyTone-Fe plates containing iron supplements. Each passage involved picking 5 to 10 single colonies (by sterile tips with 200 μl pipet) to transfer to 500 medium in a U-bottom shaped well of a 96-well plate. Each was mixed and a 50 μl cell suspension was placed and evenly spread on MF media plate. After the third passage, MF media was inoculated with each culture in a 10 ml liquid broth inoculating mini-bioreactor. The strain growth was measured by OD₅₉₀.

After attaining 2.5±0.3 OD₅₉₀, culture batches were divided into two portions. One portion used for making cell bank and the other portion was terminated by treatment with sodium deoxycholate followed by cell separation by centrifugation. Culture supernatants were enzyme treated. Post-enzyme supernatants were concentrated with 100k spin filters and the retentate collected. This retentate was analyzed by QC for the polysaccharide content per ml of broth.

Each strain was treated identically and strains streaked from working stocks to trypticase soy agar plates with 5% sheep blood in the presence of optochin disks. These plates were incubated for 16 hours at 37° C. with 5% CO₂. Colony growth was confirmed by optochin discs, agglutination, Gram staining and colony morphology.

A single strain from each colony was passaged to meat-free media agar plates containing iron supplements. A total of three passages were conducted from single colonies on veggie [meat free media] plates. Plates were incubated for 24 hours at 37° C. with 5% CO₂.

Composition of PNU-Fe Medium

Plates were prepared in a 1 L batch, giving 40 plates. All components are autoclaved prior except sodium pyruvate, ferrous sulfate and ferric citrate. The pyruvate supplement prepared at 1% and iron supplements are prepared at 0.4% stocks, 0.2 μm filtered and aseptically added to the autoclaved rest of the recipe prior plates being poured (percentages given are w/v).

IVT PNU-Fe broth was prepared according to standard protocols based on the composition below. The broth was prepared from 20 mL of each of the 50× stock salt solutions, 100 mL of the 50× Hi-Soy solution, and 100 mL of the 10× sugar stock solution. The total volume was brought to 1.0 L with Milli-Q water. Hi-Soy is a highly soluble, multi-purpose, enzymatic hydrolysate of soy meal (commercially available from Sigma-Aldrich). The salt and soy components were autoclaved for sterility whereas the sugar stock was 0.2 μm filtered.

PNU-Fe Composition
Autoclaved components
   NaCl, Final concentration: 2.0 g/L
   MgSO₄, Final concentration: 0.5 g/L
   KH₂PO₄, Final concentration: 0.7 g/L
   CaCl₂, Final concentration: 0.02 g/L
   Hi-Soy, Final concentration: 4.0 g/L
Sugar components
   D-Glucose, Final concentration: 10.0 g/L
   L-Cysteine, Final concentration: 0.2 g/L
   Thiamine HCl, Final concentration: 0.02 g/L
   Yeast Extract, Final concentration: 5.0 g/L
Supplements:
Ferrous sulfate, Final concentration: 0.004% [w/v]
Ferric citrate, Final concentration: 0.004% [w/v]
Sodium pyruvate: 0.1% [w/v]

Composition of SoyTone-Fe medium:

| | |
|---|---|
| Soy tone | 1.0% |
| Tryptone Substitute Atholate | 0.5% |
| Glucose | 1.0% |
| Veggie yeast extract | 0.5% |
| NaCl | 1.0% |

Supplements:

| | |
|---|---|
| Sodium pyruvate | 0.1% |
| Ferrous sulfate | 0.004% |
| Ferric citrate | 0.004% |

### Example 2

(a) Day-0 with Trypticase Soy Agar (TSA) Plates with 5% Sheep Blood

All the 20 strains mentioned above showed good overnight growth on blood agar plates. After streaking, optochin disks were placed on the peripherals of the streaks. These plates were incubated for 16 hours at 37° C. with 5% CO₂. Colony growth was confirmed by optochin disc zone of inhibition, agglutination, Gram staining and colony morphology.

All serotypes showed normal confluent growth on blood agar plates. Optochin disks and Gram staining was positive. Microscopic morphological observation confirmed the purity.

(b) Liquid Medium Growth and Seed (Glycerol Stock) Preparation

After the third passage growth were inoculated 20 ml liquid PNU-Fe broth in 50 ml [Falcon conical bottom] tubes and incubated at 37° C. with 5% CO₂. After attaining 0.25 to 0.3 OD₆₂₀.

The growth was harvested and the pellet was re-suspended in seed medium containing 15% glycerol. Seed stocks (5-9 vials 1 ml) were prepared and stored −80° C. until used. The seed vials of 23F, 7F, 6B, 15B, 12F, 4, 14, 8, 5, 9V, 18C, 3, 33F, 22F, 6A and 19F were tested for their growth in PNU-Fe to ensure the inoculum growth before mini-bioreactor inoculation.

(c) Mini-Bioreactor Fermentation

Assembly and sterilization of Mini-Bioreactor was performed according to standard protocols as was fermentation growth conditions. The pH was monitored (7.2) along with growth (OD₅₉₀) of the culture. When OD₅₉₀ reached 1±0.3, the feed pump was started for all serotypes.

After (attaining 2.5±0.3 OD₅₉₀) the 200 ml batch fermentation is complete, culture was divided into two parts. One 100 ml portion of the 200 ml culture was used to prepare the seed bank. Cultures were withdrawn into two sterile 50 ml conical Falcon tubes and centrifuged at 4000 g [at 10000 g for PNU3] for 25 minutes at 4° C. Supernatant was decanted without disturbing the cell pellet. Cells of the pellet were resuspended in 15% glycerol medium prepared according to standard protocols to achieve OD 2.50. Required volume of 15% glycerol media=(final OD of culture)×(final supernatant volume)/2.5. The other 100 ml portion was killed by 0.15% DOC treatment at 37° C. for 30 min and used for estimation of polysaccharide yields after partial down-stream purification described below.

(d) Down-Stream Purification
The above DOC treated culture was centrifuged at 11k for 40 minutes for cell debris separation.
The supernatant was collected, buffered (to a final concentration 20.0 mM Tris, 2.0 mM MgCl₂, pH 8.0) and then treated sequentially with a nuclease and a proteinase.
Nuclease treatment: 4 hours at 37° C., shaking at 150 RPM.
Proteinase treatment: After nuclease incubation has finished proteinase treatments for 16 hours at 37° C., shaking at 150 RPM.
Concentration by 100K spin filtration.
A 45 mL (store the remaining 55 ml at 2-8° C.) aliquot of enzyme treated supernatant was concentrated using a 100K centrifugal spin-filter. Each time 15 ml enzyme treated culture supernatant was topped on the 100k spin filter and spun at 5000 RPM for 30 min at 4° C. on a tabletop centrifuge. Retentate was spin washed with 5 ml of 150 mM NaCl and a final 1 ml normalized retentate volume was collected. This sample was submitted to QC for anthrone assay, multiplex analysis and nephelometry for serotype specific polysaccharide quantity.

### MiniBio Fermentation

6A seed growth PNU-Fe and blood agar plates. 1 ml meat free (passage) seed was inoculated in to 9 ml PNU-Fe liquid medium (pH7.2) in a 50 ml conical tube and incubated for 4 h shaking at 150 rpm in 37° C. incubator with 5% CO₂. Mean time using 250 ml vessel mini-bioreactor was assembled. pH probe was calibrated and inserted into bioreactor and processed for dry cycle steam sterilization. Later a 90 ml PNU-Fe liquid medium was aseptically transferred into the vessel. A 10 ml aliquot of base was aseptically transferred to the designated aseptic 15 ml tube connected to the bioreactor and ensured the flow (priming) in the tubing from container to the bioreactor.

Using My-control and Bioexpert software that run Applikon mini-biofermentation, arrive the set parameters of temperature (37±0.5° C.), pH 7.2 and stirring 150 RPM. Then inoculated (with 4 h conical tube grown inoculum) mini-bio vessel with sterile syringe and needle through aseptic septal port of the bioreactor. 1 ml sample was aseptically drawn from the bioreactor to obtain zero hour OD after inoculation. Allowed the fermentation process to continue at the set points. OD is obtained hourly periods until culture reached 1.2 to 1.7 OD. Each time point sample also taken on a microscopic slide.

Gram staining of these samples performed to ensure the purity of the culture during fermentation stages.

Meat Free Media PNU Seeds Identity by Colony Immunoblots

Meat free media passaged Pneumococcus seeds were cultured on blood agar and meat free agar (PNU-Fe) plates overnight. Strains were blotted from each plate onto nitrocellulose membranes. The blotted membranes were dried before being processed. Each membrane was blocked with a 2% BSA solution in PBS buffer prior to incubation with their respective primary antibodies (dilution from serum 1:500). After primary antibody incubation membranes were washed with 0.1% Tween 20 in PBS buffer. After washing membranes were incubated with HRP anti-rabbit secondary antibody (dilution from serum 1:500). Membranes were visualized using an HRP kit.

Primary Antibodies: Pneumococcal Antisera, Statens were commercially obtained (Serum Institute of India Pvt. Ltd., India). Secondary Antibodies: Anti Rabit IgG (H+L), HRP Conjugate were commercially obtained (Cat #20320, Lot #AD1527-L, Alpha Diagnostics).

Meat free agar media passaged Pneumococcal seeds were grown on blood and meat free PNU-Fe plates and loop streaks were processed for immunoblot to confirm their identity. Applikon miniBiofermentation set up (for 100 ml meat-free medium) was used for inoculation.

In the Mini-Biofermentation procedure, cultures were grown until they attain OD 590 of 1.2 to 1.7 to facilitate mid-log phase polysaccharide yields from meat free passaged cultures. Different strains reached optimal OD₅₉₀ of 1.2 to 1.7 in 4 to 6 h time with slightly different times. Fermentation batches were terminated using 0.15% DOC treatment. DOC treated broths were processed for partial purification of polysaccharides as described above.

The results show that both PNU-Fe, and SoyTone-Fe meat-free liquid media supported the pneumococcal growth. On plate media PNU-Fe with tryptone substitute atholate showed greater growth. Iron supplementation enriched the PNU regular medium composition. Glycerol seeds were successfully prepared using meat-free agar medium passage on all the strains mentioned above. The seeds were tested for MiniBio-fermenter growth and subsequently broths were estimated for their polysaccharide yields gave comparable yields (see Table 1) to that of previously observed blood agar passage seeds.

TABLE 1
PNU14 meat free seed growing in MiniBioreactor
MiniBio fermentation PS yield estimations

| Strain# | PS yield μg/ml |
|---|---|
| 6A | 201.33 |
| 19F | 204.20 |
| 33F | 167.56 |
| 12F | 119.75 |
| 7F | 233.34 |
| 4 | 390.09 |

Studies are continued to confirm the MiniBio fermenter growth and comparative polysaccharide expression and yields of these strains grown in meat-free media.

An experiment was conducted to understand the effect of additional PNU media supplementation by either: 1) Atholate alone, or 2) Atholate+Pyruvate+ iron supplements to existing PNU regular medium. Atholate is a blend of plant protein hydrolysates that matches the performance and nutritional characteristics of standard casein hydrolysate (commercially available from Athena Environmental Sciences, Inc., Maryland, USA).

Strain/vial used: PNU19F WSL[working seed lot] 1 ml volume for inoculation

Medium 1: Regular PNU medium+0.5% Tryptone subst. atholate pH 7.2 (without supplements).

Medium 2: Regular PNU medium+0.5% Tryptone subst. atholate pH7.2 (plus supplements: sodium pyruvate 0.1%+Fe⁺² 0.004%+Fe⁺³ 0.004%)

Mini-fermenter parameters were identical for both media growth (150 rpm, pH7.2 was maintained during growth, temperature 37.5° C.). Fermentation batches were terminated by adding 0.15% DOC to fermenters. Results over about a 3-6 hour period are shown in Table 2.

TABLE 2
Readings at QD 590 nm

| Hour | Medium 1 | Medium 2 |
|---|---|---|
| 3 h | 0.8 | 0.57 |
| 4 h | 2.46 | 3.07 |
| 5 h | 3.63 | 4.90 |
| 5 h 45 m | 4.23 | 6.13 |
| 6 h 15 m | 4.10 | 5.95 |

Lot of viscosity and coagulation was seen in both, but with large excess in Medium 2. In the (0.15%) DOC presence, directly added enzyme buffer and enzyme for overnight 37° C. treatment to facilitate easy centrifugation, to separate cell debris to obtain 200 ml supernatant.

Proteinase treatment followed by 100 Kd TFF and concentration steps were as per existing PNU downstream protocol.

Finally, 200 ml supernatant was concentrated to 75 ml from which lml samples were submitted to QC. Polysaccharide, protein and nucleic acid content were determined for each medium. Results are shown in Table 3.

TABLE 3
Crude PS QC results

| Result | Medium 1 | Medium 2 |
|---|---|---|
| Polysaccharide | 1284.4 μg/ml | 2720.0 μg/ml |
| | or 482 mg/L | or 1.02 g/L |
| Protein content | 2.9% | 2.9% |
| Nucleic acid content | 0.05% | 0.05% |

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. It is intended that the specification and examples be considered exemplary only with the true scope and spirit of the invention indicated by the following claims. Furthermore, the term “comprising of” includes the terms “consisting of” and “consisting essentially of.”

1. A composition comprising one or more salts; a magnesium salt; a calcium salt; soy meal; a saccharide; at least two amino acids; yeast extract; a ferrous or ferric salt; and pyruvate, wherein the composition contains no animal products.

2. The composition of claim 1, wherein the one or more salts comprises sodium chloride.

3. The composition of claim 1, wherein the magnesium salt comprises magnesium chloride or magnesium sulfate.

4. The composition of claim 1, wherein the calcium salt comprises calcium chloride or calcium sulfate.

5. The composition of claim 1, wherein the soy meal comprises an enzymatic hydrolysate of soy meal.

6. The composition of claim 1, wherein the at least two amino acids comprise cysteine and thiamine.

7. The composition of claim 1, wherein the saccharide comprise glucose.

8. The composition of claim 1, wherein the ferrous or ferric salt comprise ferrous sulfate or ferric citrate.

9. The composition of claim 1, which is an aqueous solution or a dry powder.

10. The composition of claim 9, wherein the aqueous solution comprises from about 1-5 g/L of the one or more salts; from about 0.1 to 2.0 g/L of the magnesium salt; from about 0.001 to 0.1 g/L of the calcium salt; from about 2-10 g/L of the soy meal; from about 5-20 g/L of the saccharide; from about 0.001 to 0.1 g/L of the at least two amino acids; from about 1-10 g/L of the yeast extract; from about 0.0001-0.001% of the ferrous or ferric salt; and from about 0.01-1.0% of the pyruvate.

11. The composition of claim 1, wherein the animal products excluded are obtained or derived from a mammal.

12. The composition of claim 1, wherein the products excluded that are obtained or derived from a mammal are fetal bovine serum, bovine serum, caprine serum, and/or equine serum.

13. A method for culturing a microorganism comprising:
obtaining a sample of the microorganism; and
contacting the microorganism to a medium comprising the composition of claim 1.

14. The method of claim 13, wherein the microorganism comprises Streptococcus pneumoniae.

15. A composition comprising one or more salts; soy meal; a saccharide; yeast extract; a plant protein hydrolysate, a ferrous or ferric salt; and pyruvate, wherein the composition contains no animal products.

16. The composition of claim 15, wherein the one or more salts comprises sodium chloride.

17. The composition of claim 15, wherein the soy meal comprises an enzymatic hydrolysate of soy meal.

18. The composition of claim 15, wherein the saccharide comprise glucose.

19. The composition of claim 15, wherein the yeast extract comprises a vegetable yeast extract.

20. The composition of claim 15, wherein the plant protein hydrolysate comprises atholate.

21. The composition of claim 15, wherein the ferrous or ferric salt comprise ferrous sulfate or ferric citrate.

22. The composition of claim 15, which is an aqueous solution or a dry powder.

23. The composition of claim 22, wherein the enzymatic hydrolysate of soy meal is at a concentration of from about 0.5-10%, the polysaccharide is at a concentration of from about 0.5-5%, the vegetable yeast extract is at a concentration of from about 0.1-10%, the plant protein hydrolysate is at a concentration of from about 1-10%, the ferrous or ferric salt is at a concentration of from about 0.001-0.01%, and the pyruvate is at a concentration of from about 0.01-1.0%.

24. A method for culturing a microorganism comprising:
obtaining a sample of the microorganism; and
contacting the microorganism to a medium comprising the composition of claim 15.

25. The method of claim 24, wherein the microorganism comprises Streptococcus pneumonia.
